(19) United States
(12) Patent Application Publication
(10) Pub. No.: US 2010/0185093 A1
(43) Pub. Date: Jul. 22, 2010

(54) SYSTEM AND METHOD FOR PROCESSING A REAL-TIME ULTRASOUND SIGNAL WITHIN A TIME WINDOW

(76) Inventor: James Hamilton, Brighton, MI (US)

Correspondence Address: SCHOX PLC, 500 3rd Street, Suite 515, San Francisco, CA, 94107, US

(21) Appl. No.: 12/688,787

(22) Filed: Jan. 15, 2010

Related U.S. Application Data
(63) Continuation of application No. 12/625,875, filed on Nov. 25, 2009.
Application No. 12/625,875 is a continuation of application No. 12/625,885, filed on Nov. 25, 2009.
(60) Provisional application No. 61/145,710, filed on Jan. 19, 2009.
(60) Provisional application No. 61/153,250, filed on Feb. 17, 2009.

(51) Int. Cl. A61B 8/14

(52) U.S. Cl. 600/443

(57) ABSTRACT

A method for acquiring and processing 3D ultrasound data including acquiring partial 3D ultrasound data. The partial 3D ultrasound data is composed of partial 3D ultrasound data frames that are collected by collecting an ultrasound target plane and collecting at least one ultrasound offset plane. The method additionally includes processing the partial 3D ultrasound data.

### CROSS-REFERENCE TO RELATED APPLICATIONS

This application is a continuation of prior application number U.S. patent Ser. No. 12/625,875 filed on Nov. 25, 2009 and entitled “Method and System for Acquiring and Transforming Ultrasound Data” and U.S. patent Ser. No. 12/625,885 filed on Nov. 25, 2009 and entitled “Dynamic Ultrasound Processing Using Object Motion Calculation”, which are both incorporated in their entirety by this reference.

This application also claims the benefit of claims priority to U.S. Provisional Ser. No. 61/145,710 filed on Jan. 19, 2009 and entitled “Dynamic Ultrasound Acquisition and Processing Using Object Motion Calculation” and U.S. Provisional Ser. No. 61/153,250 filed on Feb. 17, 2009 and entitled “System and Method for Tissue Motion Measurement Using ₃D Ultrasound”, which are both incorporated in their entirety by this reference.

This application is related to U.S. patent Ser. No. 11/781,212 filed on Jul. 20, 2007 and entitled “Method of Tracking Speckle Displacement Between Two Images”, (2) U.S. patent Ser. No. 11/781,217 filed on Jul. 20, 2007 and entitled “Method of Modifying Data Acquisition Parameters of an Ultrasound Device”, (3) U.S. patent Ser. No. 11/781,223 filed on Jul. 20, 2007 and entitled “Method of Processing Spatial-Temporal Data Processing”, and (4) U.S. patent Ser. No. 12/565,662 filed on Sep. 23, 2009 and entitled “System and Method for Flexible Rate Processing of Ultrasound Data”, which are all incorporated in their entirety by this reference.

### STATEMENT REGARDING FEDERALLY SPONSORED RESEARCH OR DEVELOPMENT

This invention was supported by a grant from the National Heart, Lung, and Blood Institute (#5R44HL071379), and the U.S. government may therefore have certain rights in the invention.

### TECHNICAL FIELD

This invention relates generally to the medical ultrasound field, and more specifically to a new and useful method and system for acquiring and processing 3D ultrasound in the ultrasound data acquisition and processing field.

### BRIEF DESCRIPTION OF THE FIGURES

FIG. 1 is a schematic representation the preferred embodiment of the invention;

FIGS. 2A and 2B are schematic representations of variations of the method of preferred embodiment;

FIG. 3 is a flowchart diagram of a variation of the method of the preferred embodiment including a variation of the fast-acquisition with coded transmit signals;

FIGS. 4 and 5 are graphical representations of a coded transmit signal for a preferred method of fast-acquisition;

FIG. 6 is a flowchart diagram of a variation of the method of the preferred embodiment including a variation of the fast-acquisition process with local subset acquisition;

FIG. 7 is a graphical representation of local subset acquisition for a preferred method of fast-acquisition;

FIG. 8 is a flowchart diagram of a variation of the method of the preferred embodiment including a variation of frame selection;

FIGS. 9A and 9B are graphical representations of frame selection;

FIGS. 10A and 10B are flowchart diagrams of a variation of the preferred method including multi-stage speckle tracking;

FIG. 11 is a graphical representations of multi-stage speckle tracking used for distance estimation;

FIG. 12 is a schematic representation of a preferred method of dynamic acquisition;

FIG. 13 is detailed schematic representation of a preferred method of dynamic acquisition;

FIGS. 14A and 14B are schematic representations of preferred methods of dynamic processing;

FIGS. 15A-15C are detailed schematic representations of variations of a preferred method of dynamic processing; and

FIG. 16 is a schematic diagram of the preferred embodiment of the invention.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

1. Method for Acquiring and Processing Partial 3D Ultrasound

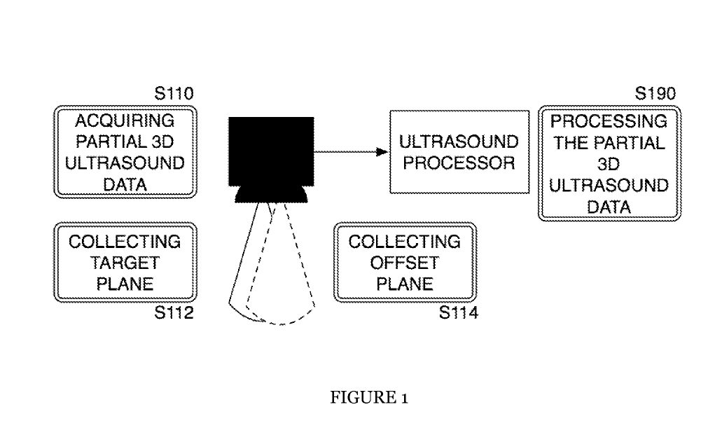

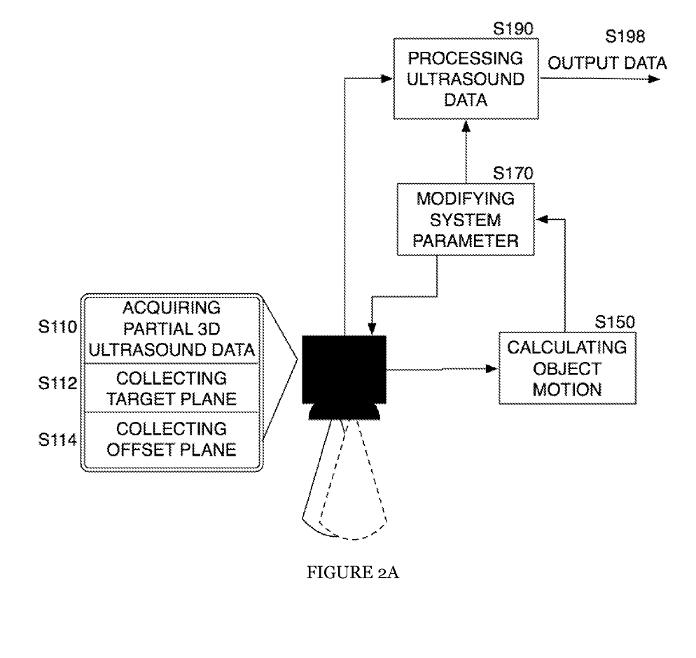

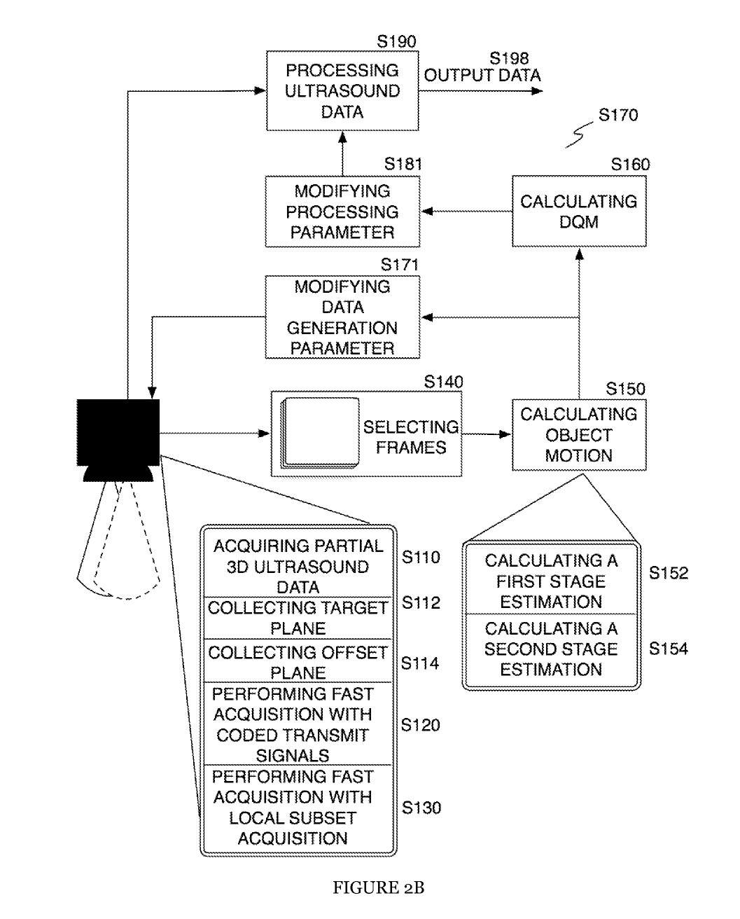

As shown in FIG. 1, the method for acquiring and processing partial 3D ultrasound of the preferred embodiment includes acquiring partial 3D ultrasound data S110 (which preferably includes the sub-steps of scanning a target plane S112 and scanning at least one offset plane S114) and processing ultrasound data related to the partial 3D ultrasound data S190. The method functions to acquire partial 3D volume of data that is substantially easier to process than normal 3D data due to a reduced volume size of the partial 3D data. Additionally, the method preferably includes calculating object motion from the collected ultrasound data S150. The partial 3D volume of data preferably enables the 3D motion tracking benefits of normal 3D ultrasound, but measured in a 2D plane. As another addition, the preferred method may include modifying system parameters based on object motion S170, as shown in FIG. 2A. Parameters may include data generation parameters S171 (i.e., dynamic acquisition) and/or processing parameters S181 (i.e., dynamic processing) as shown in FIG. 2B. Several additional alternatives may be applied to the method such as multi-stage speckle tracking, fast acquisition of data with coded transmit signals, fast acquisition of data with frame subset acquisition, frame selecting, and/or any suitable process that may be used with partial 3D data, as shown in FIG. 2B. The variations of the preferred embodiment may additionally be used in any suitable order, combination, or permutation.

Step S110, which includes acquiring partial 3D ultrasound data, functions to generate a partial 3D volume of data. A partial 3D ultrasound data set is preferably composed of partial 3D ultrasound data frames (i.e., images). The 3D ultrasound data frames preferably define a scanned volume. Step S110 preferably includes the sub-steps of scanning a target plane S112 and scanning at least one offset plane S114. Preferably, the data associated with the target plane and the offset plane are combined to form the partial 3D ultrasound data frame. Additionally, multiple offset planes may be acquired to form more detailed 3D data. Alternatively, any suitable method may be used to acquire a partial 3D volume. Temporal, partial 3D ultrasound data is preferably acquired to measure motion. Two or more partial 3D data frames are preferably used to measure motion between frames. Step S110 preferably includes the sub-steps of collecting data and preparing data. The step of collecting data functions to collect raw ultrasound data such as from an ultrasound transducer or device storing raw ultrasound data. The raw ultrasound data may be represented by real or complex, demodulated or frequency shifted (e.g., baseband data), or any suitable representation of raw ultrasound data. Preparing data functions to perform preliminary processing to convert the raw data into a suitable form, such as brightness mode (B-mode), motion mode (M-mode), Doppler, or any other suitable form of ultrasound data. Additionally, preparing data preferably includes forming the partial 3D ultrasound frames from the scans of the target plane and the offset plane(s). The acquired data may alternatively be left as raw ultrasound data, or the acquired data may alternatively be collected in a prepared data format from an outside device. In addition, pre- or post-beamformed data may be acquired. The acquired data is preferably from an ultrasound device, but may alternatively be any suitable data acquisition system sensitive to motion. The acquired data may alternatively be provided by an intermediary device such as a data storage unit (e.g. hard drive), data buffer, or any suitable device. The acquired partial 3D ultrasound may additionally be outputted as processing data and control data. The processing data is preferably the data that will be processed in Step S190. The control data may be used in motion calculation in step S150 and for system parameter modification. The processing data and control data are preferably in the same format, but may alternatively be in varying forms described above.

Sub-step S112, which includes scanning a target plane, functions to acquire a data image of material (tissue) of interest. The scanning of a target plane is preferably performed by an ultrasound transducer, but any suitable device may be used. The data image is preferably a 2D image gathered along the target plane (the plane that an ultrasound beam interrogated) or alternatively 1D data, 3D data, or any suitable data may be acquired.

Sub-step S114, which includes scanning an offset plane, functions to acquire a data image of material parallel to and offset from the target plane. The offset plane is preferably substantially parallel to the target plane and is positioned forward or backward of the target plane, preferably separated by a predetermined distance. The scanning of the offset plane is also performed in a substantially similar method as the target plane, but alternatively different ultrasound transducers, beam shapes, orientations of planes, and/or image types may be used.

Step S150, which includes calculating object motion, functions to analyze the acquired data to detect tissue movement, probe movement, and/or any other motion that affects the acquired data. Object motion preferably includes any motion that affects the acquired data such as tissue motion, tissue deformation, probe movement, and/or any suitable motion. The measured motion may be a measurement of tissue velocity, displacement, acceleration, strain, strain rate, or any suitable characteristic of probe, tissue motion, or tissue deformation. Object motion is preferably calculated using the raw partial 3D ultrasound data, but may alternatively use any suitable form of ultrasound data. At least two data frames (e.g., data images or volumes) acquired at different times are preferably used to calculate 1D, 2D or 3D motion. Speckle tracking is preferably used, but alternatively, Doppler processing, block matching, cross-correlation processing, lateral beam modulation, and/or any suitable method may be used. The motion measurements may additionally be improved and refined using models of tissue motion. The object motion (or motion data) is preferably used as parameter inputs in the modification of system parameters in Step S170, but may alternatively or additionally be used directly in the processing of Step S190.

Speckle tracking is a motion tracking method implemented by tracking the position of a kernel (section) of ultrasound speckles that are a result of ultrasound interference and reflections from scanned objects. The pattern of ultrasound speckles is fairly similar over small motions, which allows for tracking the motion of the speckle kernel within a search window (or region) over time. The search window is preferably a window within which the kernel is expected to be found, assuming normal tissue motion. Preferably, the search window is additionally dependent on the frame rate of the ultrasound data. A smaller search window can be used with a faster frame rate, assuming the same tissue velocity. The size of the kernel affects the resolution of the motion measurements. For example, a smaller kernel will result in higher resolution. Motion from speckle tracking can be calculated with various algorithms such as sum of absolute difference (SAD) or normalized cross correlation.

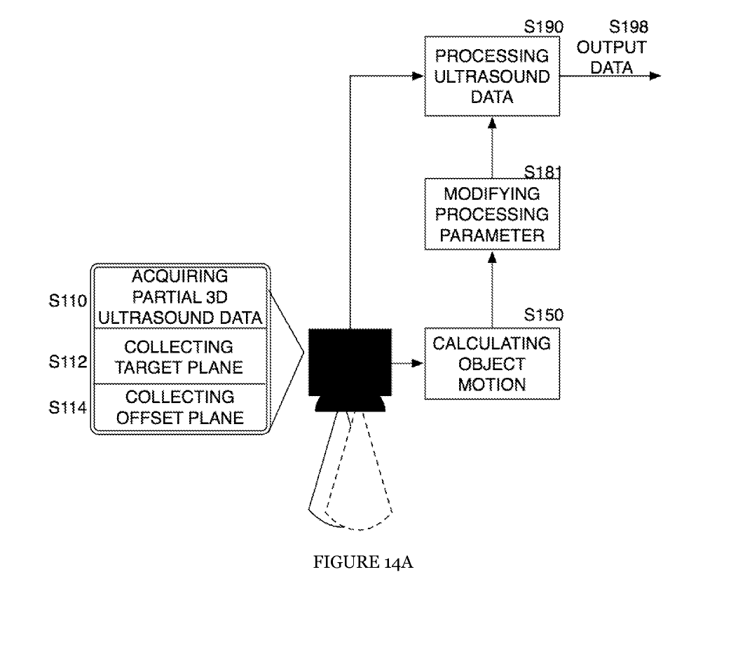

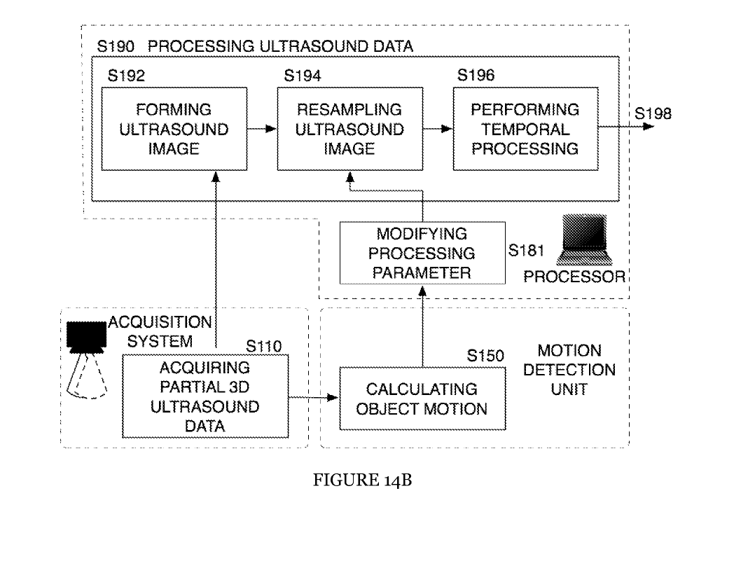

Step S190, which includes processing the partial 3D ultrasound data, functions to transform the acquired data for ultrasound imaging, analysis, or any other suitable goal. The step of processing preferably aids in the detection, measurement, and/or visualizing of image features. After the processing of the ultrasound data is complete, the method preferably proceeds in outputting the processed data (i.e., transformed data) S198. The outputted data may be used for any suitable operation such as being stored, displayed, passed to another device, or any suitable use. The step of processing may be any suitable processing task such as spatial or temporal filtering (e.g., wall filtering for Doppler and color flow imaging), summing, weighting, ordering, sorting, resampling, or other processes and may be designed for any suitable application. Preferably, Step S190 uses the partial 3D ultrasound data that was acquired in Step S110 and may additionally use any parameters that are modified in Step S170 as described below. As an example, object motion data (calculated in Step S150) may be used to automatically identify or differentiate between object features such as blood and tissue. Depending on the situation, velocity, strain, or strain-rate calculations or any suitable calculation may be optimized in step 190 to target only the object features of interest. For example, strain calculations may ignore ultrasound data associated with blood as a way to improve accuracy of tissue deformation measurements. The processing data may be raw ultrasound data (e.g., RF data) or other suitable forms of data such as raw data converted into a suitable form (i.e., pre-processed). Processing is preferably performed in real-time on the ultrasound data while the data is being acquired, but may alternatively be performed offline or remotely on saved or buffered data. As shown in FIG. 14B, processing of the partial 3D ultrasound data preferably includes the sub-steps of forming an ultrasound image S192, resampling of an ultrasound image S194, and performing temporal processing S196. The processing Steps of S190 can preferably be performed in any suitable order, and the sub-steps S192, S194, and S196 may all or partially be performed in any suitable combination.

Step S192, which includes forming an ultrasound image, functions to output an ultrasound image from the partial 3D ultrasound data acquired in Step S110. Partial 3D ultrasound data from step S110 is preferably converted into a format for processing operations. This step is optional, and is not necessary, such as in the case when the processing step is based upon raw ultrasound data. An ultrasound image is preferably any spatial representation of ultrasound data or data derived from ultrasound signals including raw ultrasound data (i.e., radio-frequency (RF) data images), B-mode images (magnitude or envelope detected images from raw ultrasound data), color Doppler images, power Doppler images, tissue motion images (e.g., velocity and displacement), tissue deformation images (e.g., strain and strain rate) or any suitable images. As partial 3D ultrasound data the ultrasound image is preferably represents a 3D volume of the object (e.g., tissue).

Step S194, which includes resampling of an ultrasound image, functions to apply the processing parameters based on the motion data to the processing of the ultrasound data. The resampling is preferably spatially focused, with temporal processing occurring in Step S196, but Step S194 and Step S196 may alternatively be implemented in substantially the same step. Ultrasound image refinements may be made using the motion data as a filter for image processing operations. For example, motion data may be used to identify areas of high tissue velocity and apply image correction (sharpening or focusing) to account for distortion in the image resulting from the motion. Additionally or alternatively, resampling of an ultrasound image may include spatially mapping data, using measurements of the spatial transformation between frames to map data to a common grid. Spatially mapping data preferably includes shifting and additionally warping images by adaptively transforming image frames to a common spatial reference frame. This is preferably used cooperatively with temporal processing of Step S196 to achieve motion compensated frame averaging.

Step S196, which includes performing temporal processing, functions to apply time based processing of successive ultrasound data images. Temporal processing preferably describes the frame-to-frame (i.e., time series) processing. Additionally, the step of performing temporal processing may be performed according to a parameter controlled by the object motion calculation. Temporal processing may include temporal integration, weighted summation (finite impulse response (FIR) filtering), and weighted summation of frame group members with previous temporal processing outputs (infinite impulse response (IIR) filtering). The simple method of frame averaging is described by a FIR filter with constant weighting for each frame. Frame averaging or persistence may be used to reduce noise. Frame averaging is typically performed assuming no motion. Temporal processing can additionally take advantage of spatial mapping of data performed in Step S194 to enhance frame averaging. For example, with a system that acquires data at 20 frames per second (i.e., 50 ms intra-frame time) and an object with an object stability time (i.e., time the underlying object can be considered constant) of 100 ms, only two frames may be averaged or processed without image quality degradation. Using measurements of the spatial transformation between frames, the data can be mapped to a common grid prior to temporal processing to compensate for object motion, providing larger temporal processing windows and ultimately improved image quality from signal to noise increase. In this example, assuming the object stability time increases by a factor of 10 (to 1 second) when the probe and object motion is removed, 20 frames can be averaged without degradation, thereby improving the signal to noise ratio by a factor greater than 3 (assuming white noise).

2. Variant Method with Fast-Acquisition of Data-Coded Transmit Signals

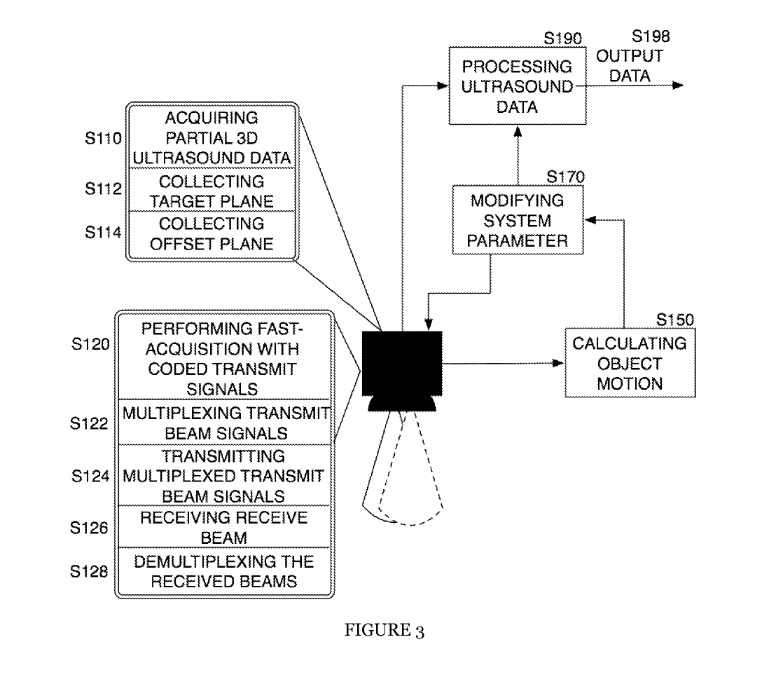

As shown in FIG. 3, the method of the preferred embodiment may additionally be used for fast-acquisition of data. The technique of fast-acquisition of data may be implemented through several variations. A coded transmit signal variation of the preferred embodiment includes the following additional steps of multiplexing a first transmit beam signal multiplexed with at least one transmit beam signal S122, transmitting the multiplexed transmit beam signals S124, receiving at least one receive beam corresponding to the transmit beam signals S126, and demultiplexing the received beams to their respective signals S128. The method of fast acquisition is preferably applied to partial 3D data collected by the methods described above, but the method of fast acquisition may alternatively be applied to full 3D or any suitable data. This variation of the preferred embodiment functions to parallelize acquisition to produce faster frame rates, but may alternatively be used for any suitable purpose. The fast acquisition steps are preferably sub-steps of Step S110 and used in Steps S112 and/or S114. However, the fast acquisition Steps may alternatively be used in place of scanning a target plane and scanning an offset plane to acquire a partial 3D volume of data.

Step S122, which includes multiplexing a first transmit beam signal multiplexed with at least one transmit beam signal, functions to multiplex the transmit beams. The step may also preferably function to allow multiple transmit beams to be transmitted simultaneously. Preferably, the transmit beam signals are modulated with orthogonal or nearly orthogonal codes. The transmit beam signals may, however, be multiplexed with any suitable modulation technique. Preferably the pulse of each transmit beam is encoded to uniquely identify it.

Step S124, which includes transmitting the multiplexed transmit beam signals, functions to transmit the multiplexed beam as transmit signals from the ultrasound system. The multiplexed transmit beam signal is preferably transmitted in a manner similar to a regular transmitted beam, but alternatively multiple ultrasound transducers may each transmit a portion of the multiplexed transmit beam signal or the signal may be transmitted in any suitable manner.

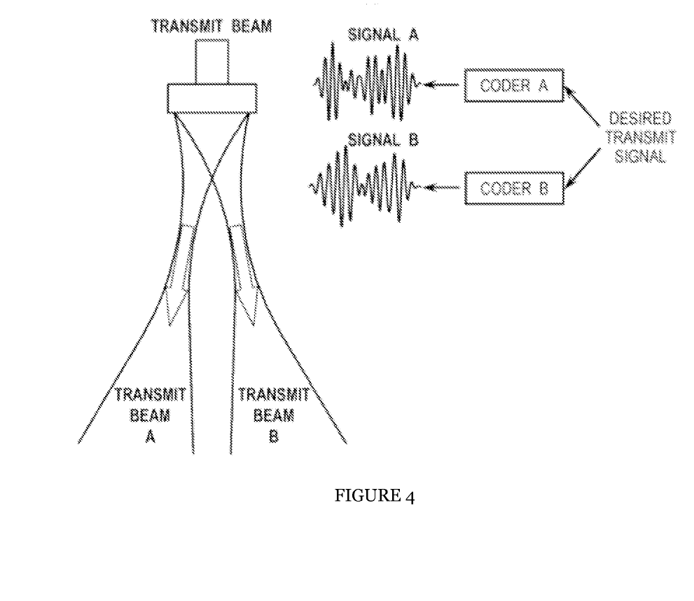

Step S126, which includes receiving at least one receive beam corresponding to each transmit beam signal, functions to detect ultrasound echoes created as the transmitted ultrasound pulse of the multiplexed transmit beam propagates. As shown in FIG. 4, these techniques of the preferred embodiment of the invention increase the data acquisition rate for ultrasound-based tissue tracking by collecting signals in multiple regions simultaneously. During signal reception, all receive beams are preferably collected simultaneously. Alternatively, the receive beams may be collected sequentially.

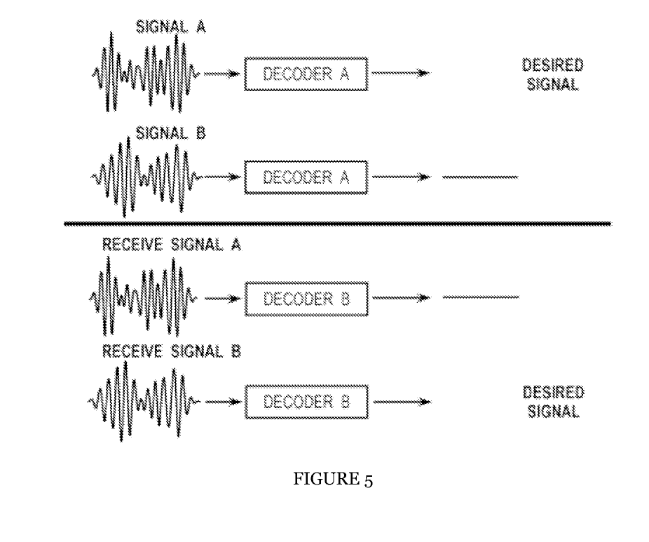

Step S128, which includes demultiplexing the received beams, functions to separate the multiplexed received beams. The processing of signals from multiple receive beams is preferably done in parallel, using coding schemes. The received beam signals are preferably demultiplexed, decoded, demodulated, filtered, or “sorted out” into their respective signals using filters specific to the transmit codes. The decoding filters preferably act only on their respective signals, rejecting others as shown in FIG. 5. To increase image quality, the codes are preferably orthogonal or nearly orthogonal.

3. Variant Method with Fast-Acquisition of Data-Frame Subset Acquisition

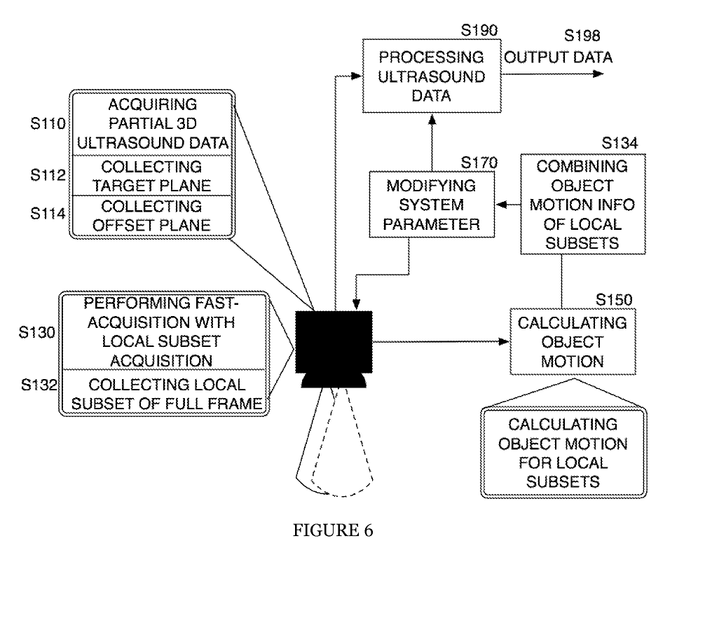

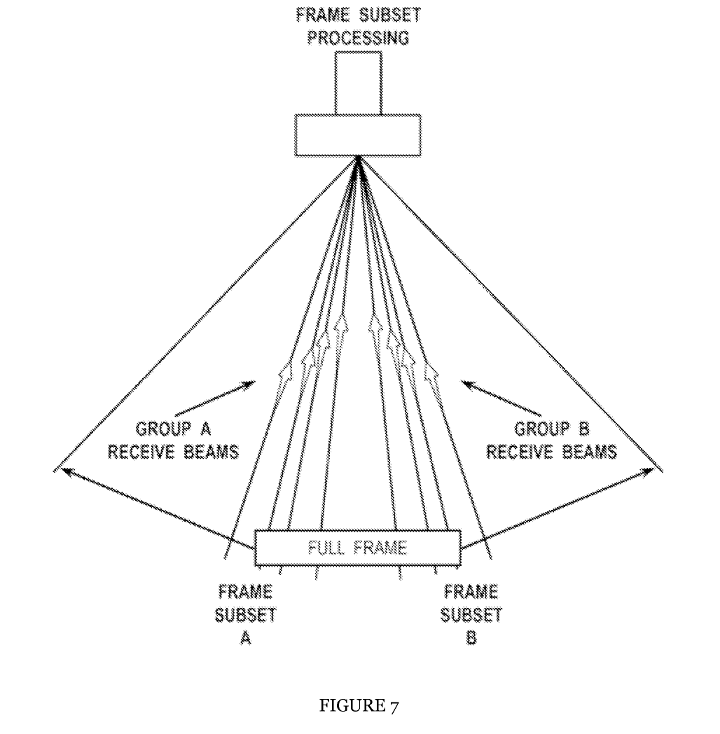

As shown in FIG. 6, as another additional or alternative variation of a technique of fast-acquisition of data, the preferred method includes collecting local subsets of the full frame at a high rate S132, calculating object motion for the local subsets in Step S150, and combining objection motion information of the local subsets (i.e., tracking results) to form full frame images at a lower rate. This frame subset acquisition variation functions to achieve high frame rates necessary for accurate tissue (speckle) tracking. As exemplified in FIG. 7, two regions, A & B, of the full frame are acquired. Beam groups A & B are used to collect these frame subsets. Each group of beams is collected at rates needed for accurate tissue tracking. Other regions of the image are preferably collected in a similar fashion. These techniques are sometimes used for colorflow imaging of blood, which also requires high local frame rates to measure high velocity blood flow. Depending on acquisition time for each beam (e.g., image depth), number of beams in a group, and local frame rate, beams from multiple groups maybe collected sequentially. For example, the collection scheme could be: beam 1 from group 1, beam 1 from group 2, beam 2 from group 1, beam 2 from group 2, and so on. As indicated above, the methods of frame subset acquisition and coded transmit signals can be combined. Preferably each subsets (portions) of a full frame are acquired and then the local tracking results are combined to form full frame images at a lower rate.

4. Variant Method with Frame Selection

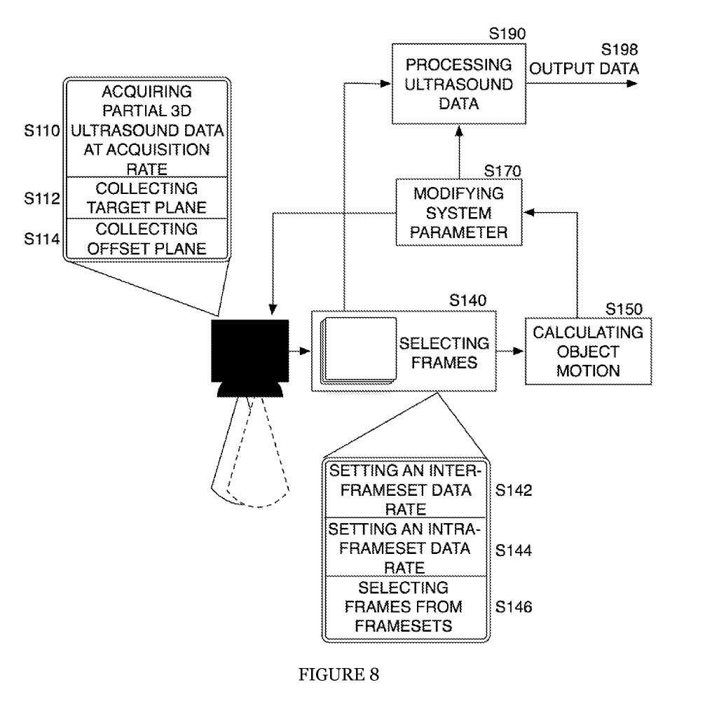

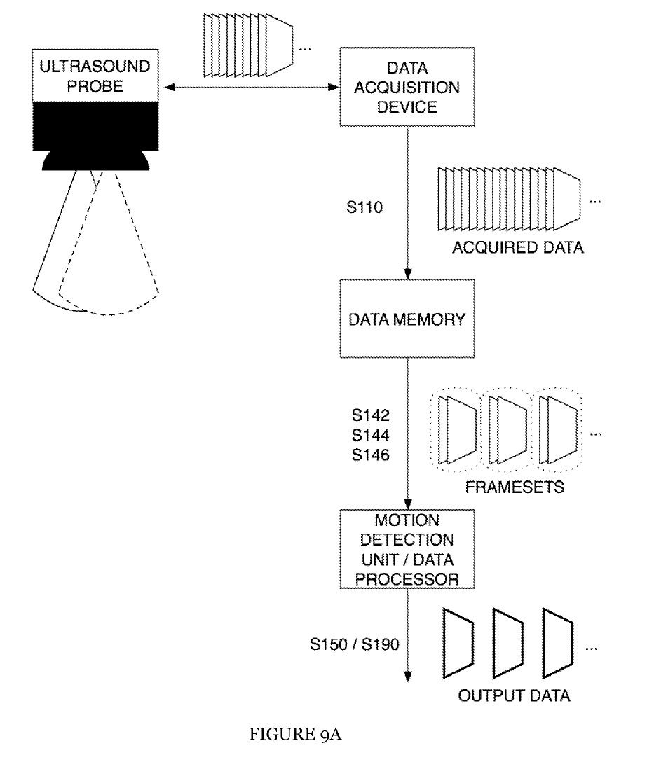

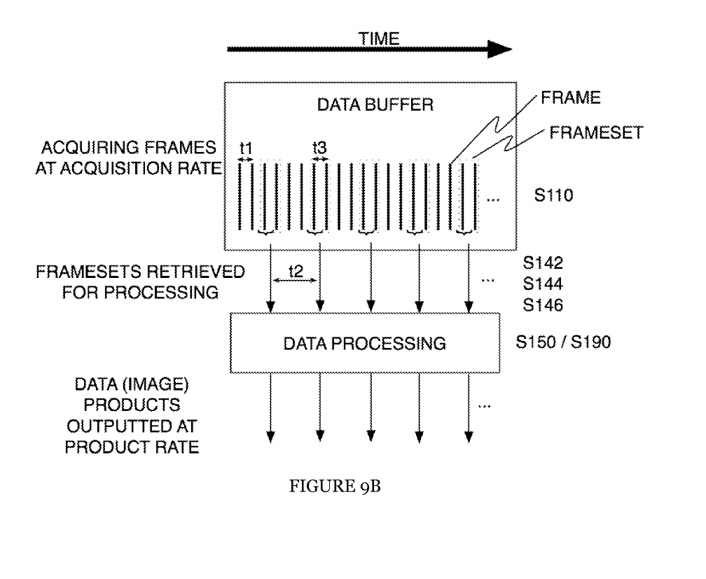

As shown in FIG. 8, the method of the preferred embodiment may additionally be used with frame selection. The step of frame selection preferably includes the substeps of capturing ultrasound data at a data acquisition rate during Step S110, setting an inter-frameset data rate S142, selecting frames to form a plurality of framesets S146, and processing the data from memory at the controlled data rates during Step S190. The preferred method of the invention may also include the step of setting an intra-frameset data rate S144. The step of frame selection functions to allow high frame rate data (the acquisition data rate) to be displayed or processed according to a second data rate (the inter-frameset data rate). As processing the partial 3D ultrasound data may include processor intensive operations, frame selection preferably allows for real-time processing to occur while preserving high frame rate data as shown in FIGS. 9A and 9B. The framesets are preferably selections of frames at a rate necessary for a processing operation, and the framesets are preferably spaced according to the inter-frameset data rate such that display or other operations (with different frame rate requirements) can be sufficiently performed. Additionally, the processing preferably occurs on raw or unprocessed ultrasound data, but may alternatively occur on pre-processed ultrasound data. Detailed analysis, additional processing, slow motion playback, fast motion playback, and/or other operations can be performed on the ultrasound data, assuming the ultrasound data is stored in memory, while still providing real-time display. While the preferred method is focused on ultrasound speckle tracking, it can also be applied to other ultrasound imaging modes in cases where decoupling of processing from acquisition rates or dynamic processing rates are desired. In one example, performing a processing task requiring data at 100 frames per second data and displaying the output at 30 frames per second, the processing requirements can be reduced to less than a third of full processing requirements without sacrificing the quality of results.

During Step S110 the partial 3D ultrasound data is preferably captured at a rate high enough to enable speckle tracking. A data acquisition rate preferably determines the time between collected ultrasound frames as indicated by t1 in FIG. 9B. For example, accurate speckle tracking of the large deformation rates associated with cardiac expansion and contraction (i.e., peak strain rates of ˜2 Hz) requires frame rates preferably greater than 100 frames per second. This frame rate is approximately 3 times greater than the frame rate needed for real-time visualization at 30 frames per second. In most cases, the frame rate required for accurate speckle tracking is greater than the frame rate needed for real-time visualization rates. The characteristics of bulk tissue motion determine visualization rates, in contrast to the interaction of ultrasound with tissue scatterers, which determines speckle-tracking rates (also referred to as intra-frameset rates). The data acquisition rate may be set to any suitable rate according to the technology limits or the data processing requirements. Maximum visualization rates are limited by human visual perception, around 30 frames per second. However, lower visualization rates may be suitable, as determined by the details of the tissue motion (e.g., tissue acceleration).

Step S142, which includes setting an inter-frameset data rate, functions to select (or sample) the frames comprising the frameset from the acquired data according to a pre-defined rate. The inter-frameset data rate is defined as time between processed framesets as indicated by t2 in FIG. 9B. Upon setting the inter-frameset data rate, Step S142 preferably includes selecting frames from acquired partial 3D ultrasound data to form a plurality of framesets S146. Step S146 functions to form the framesets for processing. The framesets are preferably spaced according to the inter-frameset data rate and any suitable parameters of the framesets. The inter-frameset data rate is preferably set to the desired output data rate such as the display rate. The inter-frameset data rate is less than or equal to the data acquisition rate. The inter-frameset data rate is preferably an integer factor of the data acquisition rate, but is otherwise preferably independent of the data acquisition rate. The acquisition rate sets the maximum rate of the inter-frameset sampling. Additionally or alternatively, parameters of the framesets may be set according to the needs of the processing step S190 or any suitable requirement. The parameters are preferably the inter-frameset data rate, but may alternatively include intra-frameset data rate, the number of frames, the number of framesets, timing of frames or framesets (such as nonlinear spacing), trigger events (from other physiological events), data compression, data quality, and/or any suitable parameter of the frameset. In one variation, the inter-frameset data rate is dynamically adjusted during acquisition (such as part of S171), preferably according to physiological motion, to better track the relative motion of the tissue (i.e. a shorter time between framesets for large tissue motion and acceleration, and a longer time between framesets for small tissue motion). In the example shown in FIG. 9B, the frameset rate (or output product rate) is one fourth (¼) of the acquisition rate.

As part of Step S190 the partial 3D ultrasound data is processed from memory at the controlled data rates. Alternatively or additionally, the processing of the partial ultrasound data at a controlled data rate may occur during the calculation of object motion S150 such as for speckle tracking. The processing is preferably individually performed on a frameset of frames. The framesets are preferably processed sequentially according to the inter-frameset data rate. The controlled data rates are preferably understood to include any set data rates governing the data rate passed to the processor, such as processing framesets at an inter-frameset data rate, processing frames of a frameset at an intra-frameset data rate, and optionally, outputting data at a product data rate. The speckle tracking is preferably performed on a frameset of two or more frames. The speckle tracking preferably processes framesets at least at rates adequate for motion measurement or visualization (e.g., 30 framesets per second), but a higher or lower frame rate may alternatively be used for other applications and requirements. For example, machine vision algorithms may require higher visualization data rates. Lower visualization data rate can be used for long term monitoring or event detection. Alternatively, any suitable processing operation may be performed such as interpolation. The processing operation preferably requires a higher frame rate than the final desired output data rate. Data is preferably output after the processing of data at a product rate. The product rate is preferably equal to the inter-frameset data rate but may alternatively be different from the inter-frameset data rate depending on the processing operation.

The preferred method also includes setting an intra-frameset data rate S144, which functions to adjust the time between frames within a frameset as indicated by t3 in FIG. 9B. The time between frames of the frameset is limited by the acquisition rate. However, while a frameset preferably comprises a pair of sequentially acquired frames, the frameset may alternatively comprise a pair of non-sequentially acquired frames acquired at the data acquisition rate (i.e. every other frame acquired at the data acquisition rate). The acquisition rate sets the maximum rate of the intra-frameset sampling. However, a variable intra-frameset data rate may be used, preferably according to physiological motion, to optimize speckle tracking performance (i.e. shorter time between frames with quickly changing speckle and longer time between frames for slowly changing speckle). A variable intra-frameset data rate is preferably set during modification of an acquisition parameter S171. The intra-frameset sampling data rate is preferably a multiple of the data acquisition rate, but is otherwise independent of the data acquisition rate. Also in the example shown in FIG. 9B, the frameset is a pair of sequentially acquired frames, and so the time between the frames of the frameset is the time between acquired frames and the intra-frameset rate is determined to be the data acquisition rate.

5. Variant Method with Multi-Stage Speckle Tracking

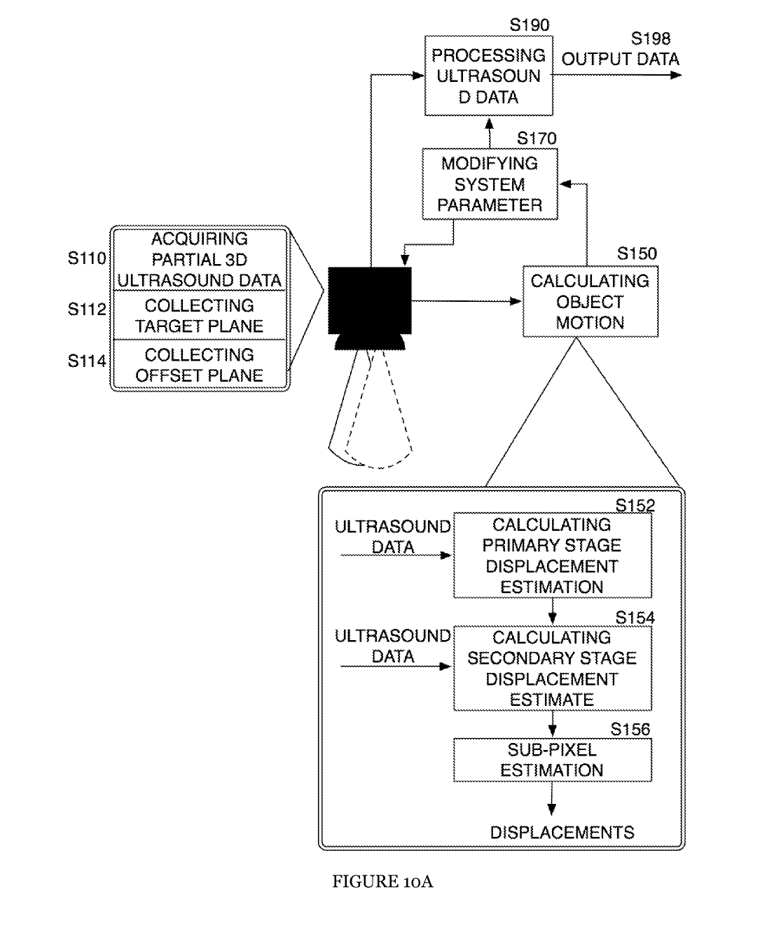

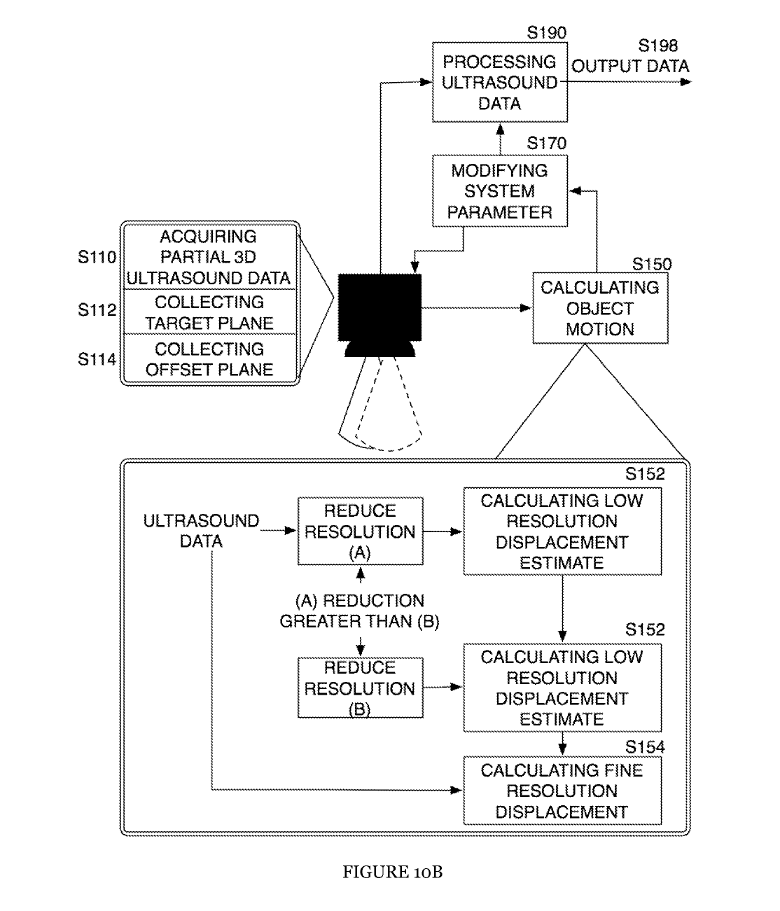

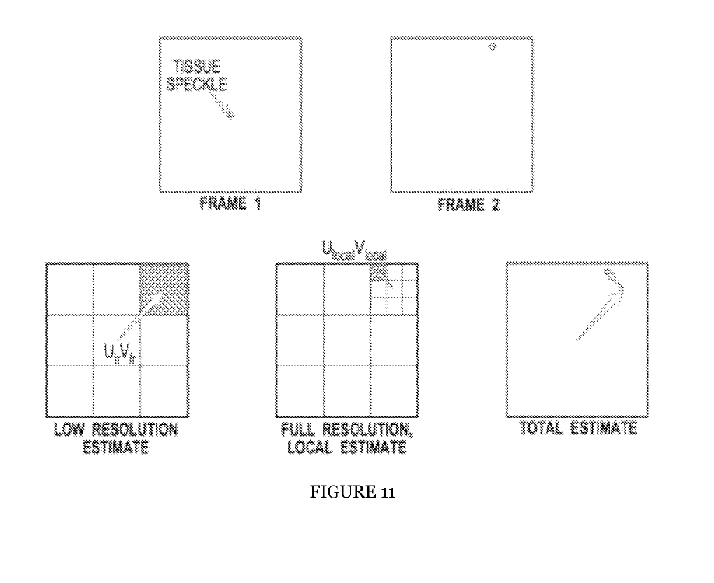

Additionally, the method of the preferred embodiment may be used for multi-stage speckle tracking, as shown in FIGS. 10A and 10B. In the multi-stage speckle tracking variation of the preferred embodiment, the step of calculating object motion S150 includes tracking speckle displacement between a first image and a second image. Step S150 of this variation preferably includes the sub-steps of calculating at least one primary stage displacement estimate S152 and calculating at least one secondary stage displacement using the first stage displacement estimate S154. Step S150 and the sub-steps of Step S150 are preferably applied to partial 3D data collected in the method described above, but Step S150 and the sub-steps of Step S150 may alternatively be applied to full 3D or any suitable data. The multi-stage speckle tracking functions to decrease the computation for image cross correlation or other suitable motion calculations. As shown in FIG. 10B, a course resolution displacement estimate is preferably used as the primary stage displacement estimate, and a finer resolution displacement estimate is preferably used as the secondary stage displacement estimate. As shown in FIG. 11, the multi-resolution variation of multi-stage speckle tracking allows for distance estimates from a low resolution image to guide a high resolution displacement estimation. This preferably decreases the computations of object motion calculation as compared to a single fine displacement estimate with no initial low resolution estimate.

Step S152, which includes calculating at least one primary stage displacement estimate, functions to calculate a lower accuracy and/or lower resolution, displacement estimation. Preferably, the primary stage displacement estimate is a coarse (low resolution and/or accuracy) displacement estimate from the ultrasound images. The coarse displacement is preferably calculated by cross correlating at least two data images, and the peak of the cross correlation function is preferably used as a coarse displacement estimate. Additionally, the resolution of the data image may be reduced prior to the estimation process. However, any method to calculate a displacement estimate may be used such as a less accurate but computationally cheaper displacement algorithm. Preferably, at least one primary stage displacement estimate is passed to step S154. The at least one primary stage displacement estimate may alternatively be passed to a successive primary stage estimation stage to perform a further primary stage displacement estimate. Each successive stage estimation stage preferably has successively more accurate and/or finer resolution results (e.g., finer resolution for the course displacement estimation) than the previous estimation stage. In the case of course resolution estimation, each coarse estimation stage may initially reduce the data image resolution to a resolution preferably finer than the previous stage. As another addition, the course displacement estimates may be upsampled to match the resolution of the following estimation stage. Any suitable number of primary stage estimations may alternatively be used before passing the primary stage estimation to Step S154.

Step S154, which includes calculating at least one secondary displacement using the primary stage displacement estimate, functions to use a primary stage displacement estimate to calculate a higher precision and/or finer resolution displacement. Primary displacement estimates are preferably used as a search offset to guide at least one finer displacement estimation, improving the computational efficiency compared to processing only using high precision and/or fine resolution stage. The primary stage displacement estimate from step S152 preferably determines regions of the original images to cross correlate. Preferably, the second stage displacement estimate is a fine resolution displacement estimate that uses a coarse resolution displacement estimate of Step S152. The fine resolution displacement is preferably the location of the peak value of the cross correlation function. More preferably, the fine resolution displacement processing provides estimates of lateral and axial motion, preferably with integer pixel accuracy. The secondary stage displacement may alternatively be computed using any suitable method such as a more accurate (and typically more computationally expensive) displacement calculation using the primary stage displacement estimate as a starting point to reduce the computation requirements.

An additional sub-step of the variation of the preferred embodiment includes calculating a sub-pixel displacement estimate Step S156 that functions to further increase the accuracy of the displacement estimate. Preferably, only the local search region of the correlation function is needed for sub-pixel displacement processing. Sub-pixel displacement calculation is preferably accomplished by parametric model fitting the correlation function from S154 to estimate the location (i.e., sub-pixel lag) of the correlation function peak, or by zero crossing of cross correlation function phase if complex image frames are used as input. Sub-pixel displacement calculation may, however, be accomplished by any suitable method or device.

6. Variant Method with Dynamic Acquisition

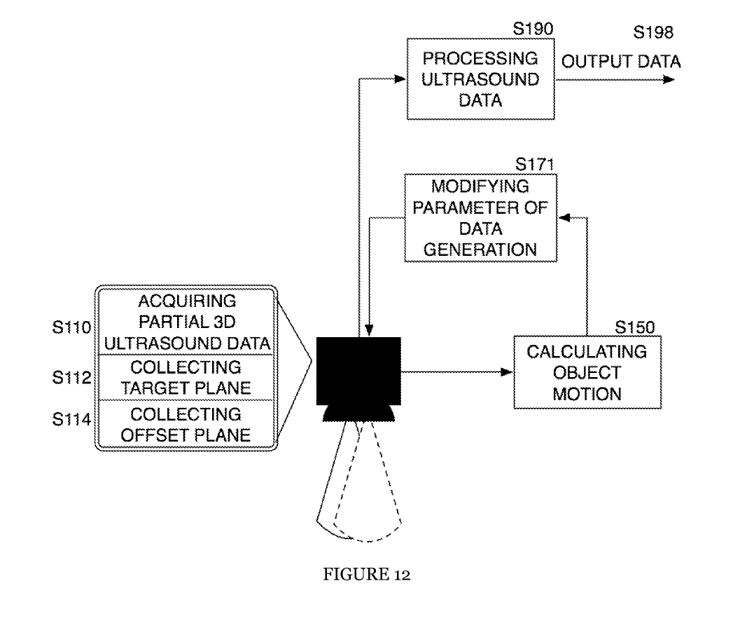

As shown in FIG. 12, the method of the preferred embodiment may additionally be used for dynamic acquisition of data as a possible variation of modifying a system parameter S170. The dynamic acquisition variation of the preferred embodiment includes the step of modifying a parameter of data generation based on object motion S171. The variation functions to optimize ultrasound data acquisition in real-time for improved ultrasound data output by adjusting the data generation process based on object motion. The calculated object motion is included in a feedback loop to the data acquisition system to optimize the data acquisition process.

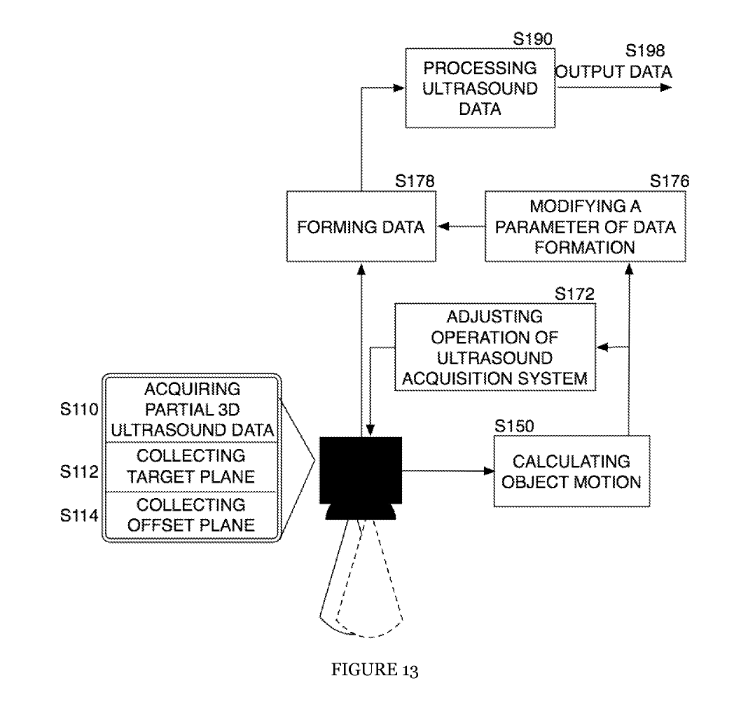

Step S171, which includes modifying a parameter of data generation, functions to alter the collection and/or organization of ultrasound data used for processing. Modifying a parameter of data generation preferably alters an input and/or output of data acquisition. Step S171 may include a variety of sub-steps. As shown in FIG. 13, the operation of the device collecting ultrasound data may be altered as in Step S172 and/or the acquired data may be altered prior to processing as in Steps S176 and S178.

Step S172, which includes adjusting operation of an ultrasound acquisition device, functions to adjust settings of an ultrasound acquisition device based on object motion data. The control inputs of the ultrasound data acquisition device are preferably altered according to the parameters calculated using the object motion. The possible modified parameter(s) of data acquisition preferably include the transmit and receive beam position, beam shape, ultrasound pulse waveform, frequency, firing rate, and/or any suitable parameter of an ultrasound device. Additionally, modifications of an ultrasound device may include modifying the scanning of a target plane and/or scanning of an offset plane. Additionally, the offset distance, number of offset planes, or any suitable parameter of partial 3D ultrasound data acquisition may be modified. Step S172 may additionally or alternatively modify parameters of any of the variations of acquiring ultrasound data such as fast data acquisition with coded transmit signals, fast data acquisition with subset acquisition, frame selection, multi-stage acquisition, and/or any suitable variation. As an example of possible modifications, previous tracking results may indicate little or no motion in the image or motion in a portion of the image. The frame rate, local frame rate, or acquisition rate may be reduced to lower data rates or trade off acquisition rates with other regions of the image. As another example, the beam spacing can be automatically adjusted to match tissue displacements, potentially improving data quality (i.e., correlation of measurements).

Additionally or alternatively, as shown in FIG. 13, the method of the preferred embodiment may include the steps modifying a parameter of data formation S176 and forming data S178. The additional steps S176 and S178 function to decouple the image (data) formation stage from other processing stages. An image formation preferably defines the temporal and spatial sampling of the ultrasound data. Steps S176 and S178 are preferably performed as part of Step S171, and may be performed with or without modifying a parameter of an ultrasound acquisition device S172 or any other alternative steps of the method 100.

Step S176, which includes modifying a parameter of data formation, functions to use the calculated object motion to alter a parameter of data formation. A parameter of data formation preferably includes temporal and/or spatial sampling of image data points, receive beamforming parameters such as aperture apodization and element data filtering, or any suitable aspect of the data formation process.

Step S178, which includes forming data, functions to organize image data for ultrasound processing. Parameters based on object motion are preferably used in the data formation process. The data formation (or image formation) stage preferably defines the temporal and spatial sampling of the image data generated from the acquired or prepared ultrasound data. The formed data is preferably an ultrasound image. An ultrasound image is preferably any spatial representation of ultrasound data or data derived from ultrasound signals including raw ultrasound data (i.e., radio-frequency (RF) data images), B-mode images (magnitude or envelope detected images from raw ultrasound data), color Doppler images, power Doppler images, tissue motion images (e.g., velocity and displacement), tissue deformation images (e.g., strain and strain rate) or any suitable images. For example, using aperture data (i.e., pre-beamformed element data) samples may be formed along consecutive beams to produce data similar to traditional beamforming.

7. Variant Method with Dynamic Processing

Additionally, the method of the preferred embodiment may be used with dynamic processing of data as a possible variation of modifying a system parameter S170, as shown in FIG. 14A. Step S181, which includes modifying processing parameter(s), functions to utilize object motion calculations to enhance or improve the data processing. The coefficients or control parameters of filters or signal processing operations are preferably adjusted according to parameter inputs that are related to the object motion calculated in Step S150. More preferably, the calculated object motion is used as the parameter inputs to modify the processing parameters. The parameter inputs may additionally or alternatively include other information such as data quality metrics discussed in further detail below. Step S181 may include variations depending on the data processing application. For example, data processing may include tissue motion calculation using speckle tracking. In this case, windows are preferably increased in size and search regions are decreased for the case of speckle tracking in a region of static tissue. Inversely, data windows are preferably decreased in size and search regions are increased for speckle tracking in regions of moving or deforming tissue. Another example of motion controlled data processing is image frame registration. In this case, motion estimates can be used to resample and align B-mode or raw data samples for improved filtering, averaging, or any suitable signal processing. Image resampling coefficients are preferably adjusted to provide frame registration. As another example, the parameter inputs may determine the coefficients or, alternatively, a new coordinate system used for processing ultrasound data such as when resampling an ultrasound image. The modified processing parameters may additionally be used in the following applications: spatial and temporal sampling of various algorithms, including color-flow (2D Doppler), B-mode, M-mode and image scan conversion; wall filtering for color-flow and Doppler processing; temporal and spatial filters programming (e.g., filter response cut-offs); speckle tracking window size, search size, temporal and spatial sampling; setting parameters of speckle reduction algorithms; and/or any suitable application.

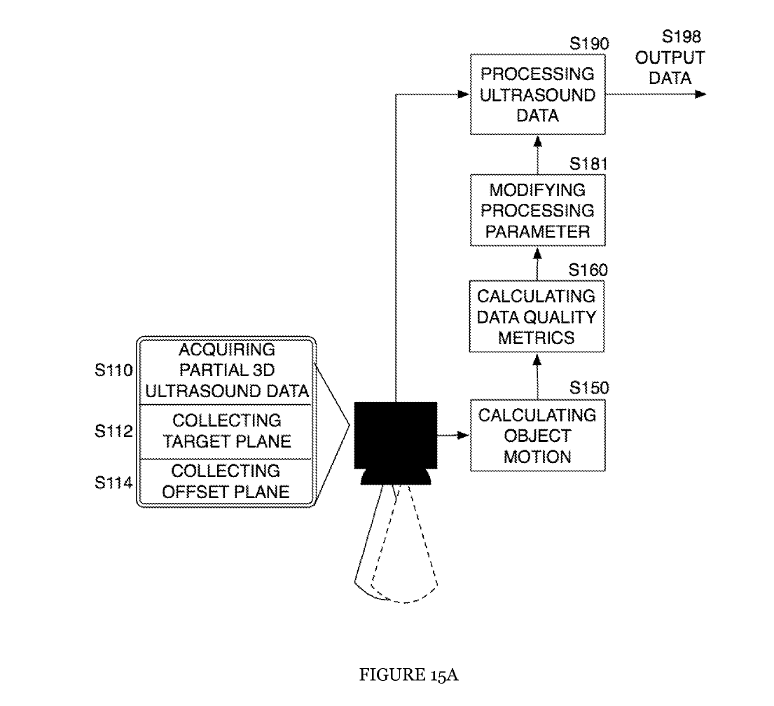

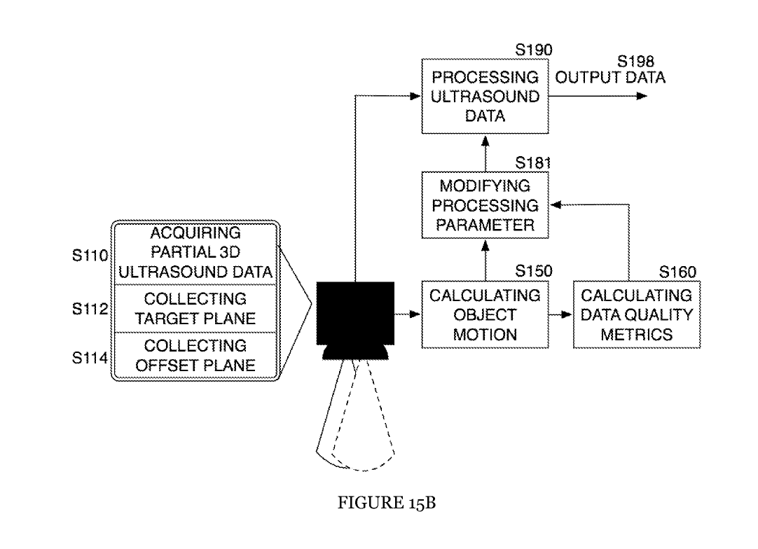

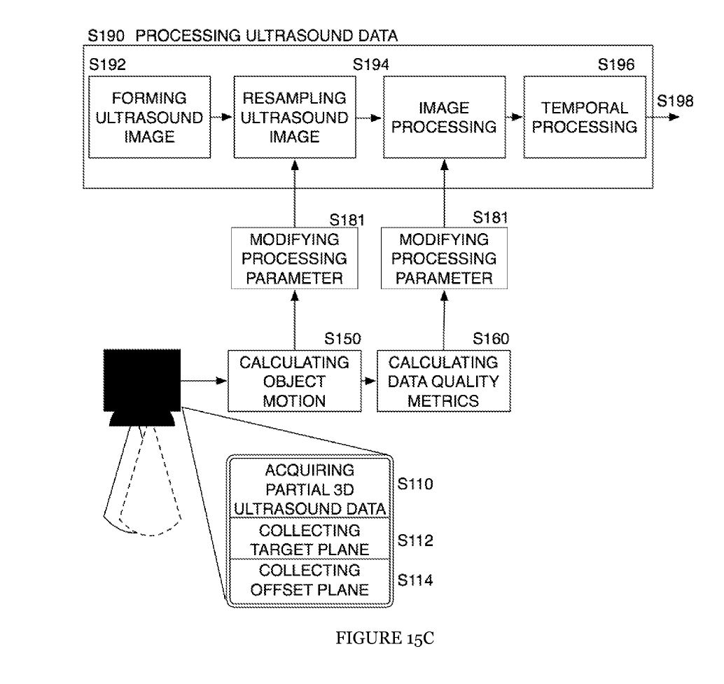

As an additional variation, as shown in FIGS. 15A, 15B, and 15C, Step S181 may be used along with a variation of the preferred embodiment including calculating a data quality metric (DQM) S160. Step S160 preferably functions to aid in the optimization of data processing by determining a value reflecting the quality of the data. The DQM preferably relates to the level of assurance that the data is valid. Data quality metrics are preferably calculated for each sample, sub-set of samples of an image region, and/or for each pixel forming a DQM map. The DQM is preferably obtained from calculations related to tissue velocity, displacement, strain, and/or strain rate, or more specifically, peak correlation, temporal and spatial variation (e.g., derivatives and variance) of tissue displacement, and spatial and temporal variation of correlation magnitude.

The data quality metric (DQM) is preferably calculated from a parameter(s) of the speckle tracking method of Step S150 and is more preferably a data quality index (DQI). Speckle tracking performed with normalized cross correlation produces a quantity referred to as DQI that can be used as a DQM. Normalized cross correlation is preferably performed by acquiring ultrasound radio frequency (RF) images or signals before and after deformation of an object. Image regions, or windows, of the images are then tracked between the two acquisitions using the cross-correlation function. The cross-correlation function measures the similarity between two regions as a function of a displacement between the regions. The peak magnitude of the correlation function corresponds to the displacement that maximizes signal matching. This peak value is the DQI. The DQI is preferably represented on a 0.0 to 1.0 scale where 0.0 represents low quality data and 1.0 represents high quality data. However, any suitable scale may be used. The DQI of data associated with tissue tend to have higher values than data in areas that contain blood or noise. As is described below, this information can be used in the processing of ultrasound data for segmentation and signal identification. The DQM is preferably used in Step S181 as a parameter input to modify processing parameters. The DQM may be used individually to modify the processing parameters (FIG. 15A), the DQM may be used cooperatively with calculated object motion to modify processing parameters (FIG. 15B), and/or the DQM and the motion information may be used to modify a first and second processing parameter (FIG. 15C).

A variation of Step S181, which includes modifying processing parameter(s), preferably utilizes object motion calculations and/or DQM to enhance or improve the data processing. The coefficients or control parameters of filters or signal processing operations are preferably adjusted according to the parameter inputs related to object motion measured in Step S150 and/or the DQM of Step S160. The modification of processing parameters may be based directly on DQM (FIG. 15A) and/or calculated object motion (FIGS. 14A and 14B). The modification of the processing parameters may alternatively be based on a combination of the processing parameters either cooperatively as in FIG. 15B or simultaneously (e.g., individually but in parallel) as in FIG. 15C.

The use of DQM preferably enables a variety of ways to control the processing of data. For example, measurements such as B-mode, velocity, strain, and strain rate may be weighted or sorted (filtered) based on the DQM. The DQM can preferably be used for multiple interpretations. The DQM may be interpreted as a quantized assessment of the quality of the data. Data that is not of high enough quality can be filtered from the ultrasound data. As an example, ultrasound derived velocity measurements for a section of tissue may suffer from noise. After filtering velocity measurements to only include measurements with a DQI above 0.9, the noise level is reduced and the measurement improves. The DQM may alternatively be interpreted as a tissue identifier. As mentioned above, the DQI can be used to differentiate between types of objects specifically, blood and tissue. Thus, the DQI can be used for segmentation and signal or region identification when processing the ultrasound data. As an example of one application, the DQM, or more specifically the DQI, may be used to determine the blood-to-heart wall boundaries and may be used to identify anatomical structures or features automatically. Processing operations may additionally be optimized by selectively performing processing tasks based on identified features (e.g., tissue or blood). For example, when calculating strain rate of tissue, areas with blood (as indicated by low DQI) can be ignored during the calculation process. Additionally, higher frame rates and higher resolution imaging require more processing capabilities. Using DQM to segment ultrasound data or images according to tissue type, tissue specific processing operations can be used to reduce processing requirements for computationally expensive processes. In this variation, computational expensive processes are performed for data of interest. Data of less interest may receive a different process or a lower resolution process to reduce the computational cost.

8. System for Acquiring and Processing Partial 3D Ultrasound

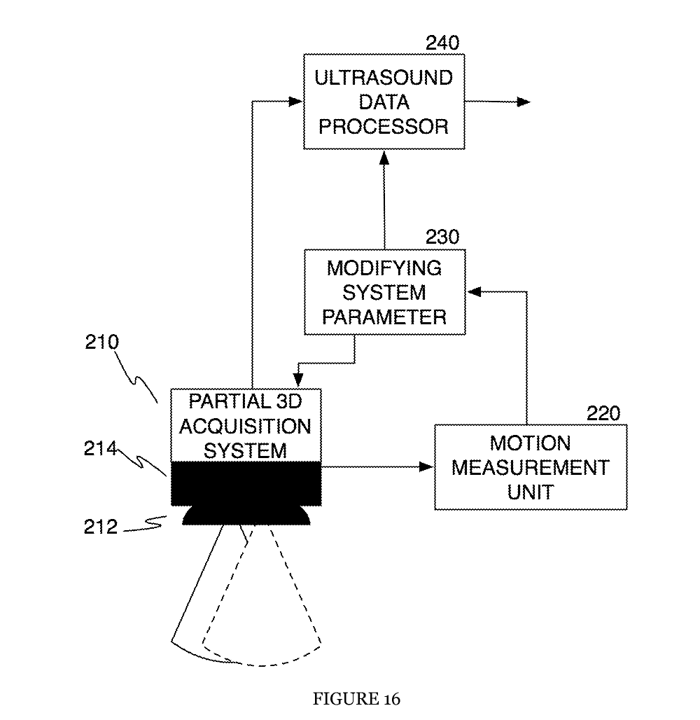

As shown in FIG. 16, the preferred system of three-dimensional (3D) motion tracking in an ultrasound system includes a partial 3D ultrasound acquisition system 210, a motion measurement unit 220, and an ultrasound processor 240. The system functions to acquire a partial 3D volume of data that is substantially easier to process due to a reduced volume size as compared to full volume 3D data. The function is to produce 3D motion measurements in a 2D plane.

The partial 3D ultrasound acquisition system 210 functions to collect a partial 3D volume of tissue data. A partial 3D volume is a volume that has one dimension with a substantially smaller size and/or resolution than the other dimensions (e.g. a plate or slice of a 3D volume). The partial 3D ultrasound system preferably includes an ultrasound transducer 212 that that scans a target plane and at least one offset plane and a data acquisition device 214. Preferably, the data collected from the target plane and the offset plane are each a two-dimensional (2D) data image. The target plane and offset plane are preferably combined to form a partial 3D volume. Acquiring at least two volumes at different times enables tissue motion to be measured in three dimensions. Multiple ultrasound transducers may be used to acquire target and offset planes. Alternatively, any suitable number of planes of ultrasound data, arrangement of transducers, and/or beam shape may be used to collect the partial 3D volume of tissue data. The data acquisition device 214 preferably handles the data organization of the partial 3D ultrasound data. Additionally the partial 3D ultrasound acquisition system 210 may be designed to implement processed described above such the fast acquisition with coded transmit signals, fast data acquisition with frame subset acquisition, frame selection, and/or any suitable process of ultrasound acquisition.

The ultrasound transducer 212 of the preferred embodiment functions to acquire ultrasound data from the target and offset plane(s). The ultrasound transducer 212 is preferably similar to ultrasound devices as commonly used for 1D or 2D ultrasound sensing, and the main ultrasound transducer 212 preferably transmits and detects an ultrasound beam. The ultrasound transducer 212 may, however, be any suitable device. A transmitted beam preferably enables the collection of data from material (tissue) through which it propagates. Characteristics of the pulse and beam are controlled by a beamformer. The target plane is preferably a 2D data image and is preferably the region interrogated by the ultrasound beam. The acquired data is preferably raw ultrasound data. Raw ultrasound data may have multiple representations such as real or complex, demodulated or frequency shifted (e.g., baseband data), or any suitable form of raw ultrasound data. Raw ultrasound data may be prepared to form brightness mode (B-mode), motion mode (M-mode), Doppler, or any suitable prepared form of ultrasound data.

The target plane of the preferred embodiment is preferably 2D ultrasound data of a plane of interest. The target plane is preferably scanned by the ultrasound transducer, but may alternatively be acquired by a dedicated device, multiple transducers, or any suitable device.

The offset plane of the preferred embodiment is preferably identical to the target plane except as noted below. The offset plane is preferably parallel to the target plane, but offset by any suitable distance. The distance is preferably identical or similar to the desired magnitude of object motion (e.g. expected tissue motion or probe motion in offset direction). Additionally, any suitable number of offset planes may be acquired.

The data acquisition device 214 of the preferred embodiment functions to organize the ultrasound data into 3D volume data. The data acquisition device 214 preferably handles communicating the data to outside devices, storing the data, buffering the data, and/or any suitable data task. The data acquisition device preferably leaves the data in a raw data form (unprocessed), but the data acquisition may alternatively perform any suitable pre-processing operations.

The motion measurement unit 220 of the preferred embodiment functions to analyze the partial 3D volume of data to detect object motion. Object motion preferably includes tissue movement, probe movement, and/or any suitable motion affecting the acquired data. Object motion is preferably calculated using the raw ultrasound data. At least two sets of data acquired at different times are preferably used to calculate 1D, 2D or 3D motion. Speckle tracking is preferably used, but alternatively, Doppler processing, cross-correlation processing, lateral beam modulation, and/or any suitable method may be used. The motion measurements may additionally be improved and refined using object motion models (e.g. parametric fit, spatial filtering, etc.). The motion measurement unit 220 may additionally calculate a data quality metric (DQM), which may be used by the ultrasound data processor or any suitable part of the system as an input variable.

Additionally, the system of the preferred embodiment includes a system parameter modifier 230. The system parameter modifier 230 preferably uses the object motion information generated by the motion measurement unit for adjusting aspects of the whole system. More preferably the system parameter modifier modifies parameters of the partial 3D ultrasound acquisition system or parameters of the ultrasound data processor. Additionally the DQM of the motion measurement unit may be used to determine the operation of the system parameter modifier.

The ultrasound data processor 240 of the preferred embodiment functions to convert the ultrasound data into another form of data. The ultrasound data processor may additionally use processing parameters determined by the system parameter modifier.

An alternative embodiment preferably implements the above methods in a computer-readable medium storing computer-readable instructions. The instructions are preferably executed by computer-executable components for acquiring and processing the partial 3D ultrasound data. The computer-readable medium may be stored on any suitable computer readable media such as RAMs, ROMs, flash memory, EEPROMs, optical devices (CD or DVD), hard drives, floppy drives, or any suitable device. The computer-executable component is preferably a processor but the instructions may alternatively or additionally be executed by any suitable dedicated hardware device. An ultrasound acquisition device as described above may additionally be used in cooperation with a computer executable component.

As a person skilled in the art will recognize from the previous detailed description and from the figures and claims, modifications and changes can be made to the preferred embodiments of the invention without departing from the scope of this invention defined in the following claims.

We claim:

1. A method for acquiring and processing 3D ultrasound data comprising:
acquiring partial 3D ultrasound data composed of partial 3D ultrasound data frames, wherein a partial 3D ultrasound data frame is collected by:
collecting an ultrasound target plane; and
collecting at least one ultrasound offset plane; and
processing the partial 3D ultrasound data.

2. The method of claim 1, wherein the offset plane is substantially parallel and displaced a set distance from the target plane, and the target plane and the at least one offset plane cooperatively combine to form a 3D volume image.

3. The method of claim 1, wherein acquiring a partial 3D ultrasound data composed of partial 3D ultrasound data frames further includes:
multiplexing a first transmit beam signal with a second transmit beam signal;
transmitting the multiplexed transmit beam signals;
receiving at least one receive beam corresponding to the first transmit beam signal and at least one receive beam corresponding to the second transmit beam signal; and
demultiplexing the received beams.

4. The method of claim 3, further comprising modulating the transmit beam signals with substantially orthogonal codes.

5. The method of claim 1, wherein acquiring a ultrasound data composed of partial 3D ultrasound data frames further includes:
collecting local subsets of a full ultrasound data frame at a high rate;
calculating object motion from the collected ultrasound data for the local subsets; and
combining objection motion information of the local subsets to form full frame images at a lower rate.

6. The method of claim 1, further comprising:
setting an inter-frameset data rate;
setting an intra-frameset data rate;
selecting frames from the acquired partial 3D ultrasound data to form a plurality of framesets at an inter-frameset and intra-frame set data rates; and
wherein processing ultrasound data is performed on the framesets.

7. The method of claim 1, further comprising calculating object motion from the acquired partial 3D ultrasound data.

8. The method of claim 7, wherein calculating object motion further includes:
calculating at least one first stage displacement estimation from a first ultrasound data frame and second ultrasound data frame; and
calculating at least one second stage displacement estimation from the first ultrasound data frame, the second ultrasound data frame, and the first stage displacement estimate.

9. The method of claim 8, wherein a first stage displacement estimation is a lower resolution displacement estimation than the second stage displacement estimation.

10. The method of claim 8, wherein a first stage displacement estimation is a lower accuracy estimation than the second stage displacement estimation.

11. The method of claim 7, further comprising modifying a system parameter based on the calculated object motion.

12. The method of claim 11, wherein modifying a system parameter includes modifying a parameter of data generation based on object motion.

13. The method of claim 12, wherein modifying a parameter of data generation includes adjusting operation of an ultrasound acquisition device that is acquiring the partial 3D ultrasound data.

14. The method of claim 12, wherein a modifying a parameter of data generation includes modifying a parameter of data formation and forming the partial 3D ultrasound data prior to processing the ultrasound data.

15. The method of claim 11, wherein modifying a system parameter includes modifying a processing parameter based on object motion.

16. The method of claim 15, further comprising calculating a data quality metric;
wherein modification of a processing parameter is additionally based on the data quality metric.

17. The method of claim 16, wherein processing partial 3D ultrasound data includes forming an ultrasound image, resampling of the ultrasound image, and performing temporal processing.

18. The method of claim 12, wherein modifying a system parameter additionally includes modifying a processing parameter based on object motion.

19. The method of claim 18, further comprising:
acquiring a partial 3D ultrasound data by performing a technique of technique of fast-acquisition of data;
setting an inter-frameset data rate;
selecting frames from the acquired partial 3D ultrasound data to form a plurality of framesets at an inter-frameset data rate;
wherein processing ultrasound data is performed on the framesets; and
wherein calculating object motion includes:
calculating at least one first stage displacement estimation from a first ultrasound data frame and second ultrasound data frame; and
calculating at least one second stage displacement estimation from the first ultrasound data frame, the second ultrasound data frame, and the first stage displacement estimation.

20. A system for acquiring and processing 3D ultrasound data comprising:
a partial 3D Acquisition system that collects a target data plane and an offset data plane to form a partial 3D ultrasound data frame;
a motion measurement unit; and
an ultrasound processor.

21. The system of claim 20, further comprising a system parameter modifier that uses the output of the motion measurement unit to adjust settings of the system.
